(19) United States
(12) Patent Application Publication
(10) Pub. No.: US 2024/0060988 A1
(43) Pub. Date: Feb. 22, 2024

(54) THIOL VARIANTS AND ANALYTICAL METHODS THEREOF

(71) Applicant: DR. REDDY’S LABORATORIES LIMITED, Hyderabad (IN)

(72) Inventors: Rajiv Pranesh BHARADWAJ, Mallathahalli Bangalore (IN); Murali JAYARAMAN, Kancheepuram (Dist) (IN); Sakthi CHANDINI D, Chitlapakkam Chennai (IN); Aishwarya NATARAJAN, Delhi (IN); Aakash CHANDRAMOULI, Chennai (IN)

(21) Appl. No.: 18/278,571

(22) PCT Filed: Feb. 24, 2022
(86) PCT No.: PCT/IN2022/050159

§ 371 Date: Aug. 23, 2023

(30) Foreign Application Priority Data
Feb. 25, 2021 (IN) 202141007890

(51) Int. Cl. G01N 33/68; C07K 16/24

(52) U.S. Cl. CPC G01N 33/6854; C07K 16/244; G01N 2440/36; G01N 2440/34; C07K 2317/41

(57) ABSTRACT

The free thiol group present in free cysteines in recombinant therapeutic antibodies are reactive to process components and generates product variants during early stages of biosimilar development. Free thiol group present on the structural motifs, especially in the complementary determining regions (CDR), support maximal antigen binding capability. Product variants associated with these free thiol groups are detrimental for safety and efficacy of these therapeutic antibodies. Methods to identify and characterize various thiol variants an antibody composition is provided and an anti-IL-17A IgG1 composition having these thiol variants are described.

### FIELD OF INVENTION

The Biological material used in the invention was not obtained from India. The present invention is related to protein composition, particularly antibody composition, comprised of thiol variants and methods for analysis and characterisation of the thiol variants present therein.

### BACKGROUND OF THE INVENTION

Recombinant monoclonal antibodies (mAbs) have emerged as an important class of biopharmaceutical drugs. Recombinant mAbs are inherently complex and heterogenous molecules which is due to various factors like various post-translational modifications e.g glycosylation, that occur within the cell they are expressed and chemical modifications that may occur during different steps of manufacturing e.g., oxidation, deamidation. The resultant mAb variants often lead unwanted negative effect on stability and activity of these drugs. Therefore, regulatory agencies warrant that these recombinant mAb variants be well characterised to their ensure safety and intended efficacy.

Most of the recombinant mAbs are of IgG isotype and they share the evolutionary conserved disulfide bonds between the cysteine residues described for naturally human IgG. While number of intra-chain disulfide bonds in the various IgG subclasses is same, the number of inter-chain disulfide bonds varies amongst the various IgG subclasses. It is presumed that sulfhydryl group of all cysteines in IgG are in disulfide bonded state. However, presence of free sulfhydryls has been reported both in human IgGs as well as recombinant mAbs which are attributable reduction of intra-chain as well as intra-chain disulfide bonds (Liu and May, MAbs. 2012 January-February; 4(1):17-23). Further still, monoclonal antibodies have been reported wherein cysteine residues other than canonical disulfide linkage associated cysteines have been described, which have free sulfhydrl group (Gadgil et al. Anal. Biochem. 355 (2006) 165-174; McSherry et. al. MABS(2016), VOL. 8, NO. 4, 718-725).

The free sulfhydryls may result in unintended disulfide bond formation leading to dimerization and aggregate formation. The free cysteines are also susceptible to chemical modification such as cysteinylation, cystinylation and glutathionylation. Such chemical modification can affect the biophysical and biochemical properties in some cases the bioactivity of the protein. Chemical modification of free cysteines can be especially problematic if these are present in complementarity-determining regions (CDRs) of a therapeutic antibody, modification of those free cysteines could affect the efficacy of the antibody.

Secukinumab is a recombinant fully humanized monoclonal immunoglobulin G1 (IgG1)/κ antibody that selectively targets IL-17A and blocks its interaction with the IL-17 receptor. It has a non-canonical cysteine present at 97^{th} position in the light chain (CysL97) of secukinumab (WO2016103146). This cysteine is in the CDR3 region of the light chain of the antibody. Any chemical modification of these non-canonical cysteines may negatively affect the activity and stability of the antibody. Thus, it is imperative that the compositions containing secukinumab be characterized for the presence of variants that may arise due the chemical modification of this residue.

The present invention provides a workflow to characterize the thiol variants that may result due the free sulhydryl groups that may be present in an antibody molecule. Further this invention provides anti-IL-17A compositions that characterised by the presence various CysL97 variants including free cysteine variant, cysteinylated variant, gluththionylated variant, dimerized variant.

### SUMMARY OF THE INVENTION

Monitoring thiol variants is essential for the optimization of process parameters during drug development as the observed variants can affect function, stability, and efficacy of the molecule. The present invention identifies and characterizes the product-related thiol variants in monoclonal antibodies having free cysteine, which are vulnerable towards generating charge variants. Hence, the present invention can be used as a viable tool for continuous monitoring of thiol variants during cell line development, top clone selection, process optimization, scale-up and related product development domains.

The invention, in particular, discloses a method to identify and characterize the thiol variants of an antibody consisting of non-canonical free cysteine.

### DETAILED DESCRIPTION OF THE INVENTION

### Definitions

The term “about” refers to a range of values that are similar to the stated reference value to a range of values that fall within 25, 20, 19, 18, 17, 16, 15, 14, 13, 12, 11, 10, 9, 8, 7, 6, 5, 4, 3, 2, 1 percent or less of the stated reference value.

The term “antibody” as used herein encompasses whole antibodies and any antigen binding fragment (i.e., “antigen-binding portion”) or single chains or fusion protein thereof. An “antibody” refers to a glycoprotein comprising at least two heavy (H) chains and two light (L) chains inter-connected by disulfide bonds, or an antigen binding portion thereof.

The term “canonical cysteine” refers to a cysteine residues in an antibody sequence which are involved in evolutionary conserved disulfide linkages described in immunoglobulins.

The term “charged variants” refers to acidic variants and basic variants in the antibody composition.

The term “cysteinylation” refers to a protein modification that effectively converts an L-cysteine residue to S-(L-cysteinyl)-L-cysteine, forming a disulfide bond with free cysteine. The “cysteinylated variant” refers to the thiol variant comprising such bonded cysteine in their amino acid chain.

The term “free cysteine” or “unpaired cysteine” refers to a cysteine that is not involved in disulfide bonding. These include free cysteine residues resulting due to reduction of the cysteines involved in conserved disulfide linkages in immunoglobulins and native free cysteine residues that are not involved in conserved disulfide linkages described in immunoglobulins. Further “free cysteine variant” refers to the thiol variant wherein the free sulfhydryl group in “free cysteine” is not chemically modified.

The term “glutathionylation” refers to modification or protein wherein formation of disulfide bond between protein cysteines and glutathione (GSH) cysteine takes place. The term “glutathionylated variant” refers to the thiol variant comprising such bonded cysteine with glutathione.

The term “thiol variant” refers an antibody variants that may result due to chemical modification of sulfhydryl group present in free cysteine in the antibody. The exemplary chemical modifications include cysteinylation, cystinylation, glutathionylation, intramolecular or intermolecular disulfide bond scrambling. The term includes variants in which the free cysteine variants as well as dimer variants in which two antibody molecules are linked by a disulfide bond formed between the free cysteines of two antibody molecules.

The present invention discloses a method to identify and characterize thiol variants of an antibody at protein level and peptide level.

In an embodiment, the present invention provides a method to identify and characterize thiol variants of an antibody, the method comprising
sample preparation, subjecting the sample to ultra-performance liquid chromatography (UPLC), detecting the variant using high resolution mass spectrometry and identifying the variants
   wherein the thiol variant arise due to presence of free cysteines or chemical modification of free cysteines derived from either a canonical cysteine involved in disulfide linkage or a non-canonical cysteine.

In an embodiment, the present invention provides a method to identify and characterize thiol variants of an antibody, the method comprising
sample preparation, subjecting the sample to ultra-performance liquid chromatography (UPLC), detecting the variant using high resolution mass spectrometry and identifying the variants
   wherein the thiol variant arise include variants selected from free cysteine variant, cysteinylated variant, cystinylated variant, glutathionylated variant and dimer variant.

In an embodiment, the samples used in the present method are selected from intact antibody with native glycosylation, intact deglycosylated antibody, proteolytic digests of non-reduced alkylated antibody or proteolytic digests of non-reduced non-alkylated antibody molecule.

In an embodiment, the antibody molecule, to which the method of present invention is applicable, has a non-canonical cysteine residue in its light chain. In yet another embodiment, the antibody molecule has a non-canonical cysteine residue in the CDR region of the light chain. In a preferred embodiment, the antibody molecule has a non-canonical cysteine residue on CDR3 region of the light chain. In a most preferred embodiment, the antibody molecule has a non-canonical cysteine at 97^{th} position of light chain, the position being designated according to Kabat numbering scheme.

In an embodiment, the method of present invention is applicable to antibody molecules which have more than one cysteine residues that can give rise thiol variants. In yet another embodiment, the method of present invention is applicable to antibody molecules which have more than one cysteine residues that can give rise thiol variants, and wherein these multiple cysteine residue are homogenously modified or have mix of possible chemical modification. For example, an antibody molecule having a free cysteine in each of its light chain, each of the cysteine residue has same modification e.g. cysteinylation, or has different modification on each light chain e.g. cysteinylation in one light chain while glutathionylation in other light chain.

In an embodiment, the present invention provides an antibody composition comprising thiol variants wherein the thiol variants arise from presence of free cysteine variant or chemical modification of free sulfhydryl present on free cysteines derived from other wise disulfide bonded cysteines and/or non-canonical free cysteines. In another embodiment, the present invention provides an antibody composition comprising thiol variants wherein the thiol variants being selected from the group comprising free cysteine variant cysteinylated variant, cystinylated variant, glutathionylated variant, variants having intramolecular or intermolecular disulfide bond scrambling.

In an embodiment, the present invention provides an antibody composition comprising thiol variants, wherein the antibody a non-canonical cysteine residue in the CDR region of the light chain. In an embodiment, the present invention provides an antibody composition comprising thiol variants, wherein the antibody has a non-canonical cysteine residue on CDR3 region of the light chain. In a yet another embodiment, the present invention provides an antibody composition comprising thiol variants wherein the antibody has a non-canonical cysteine at 97^{th} position of light chain, the position being designated according to Kabat numbering scheme. In one embodiment, the antibody composition of the present invention is of anti-IL-17A antibody. In some embodiments the said anti-IL-17A antibody composition is comprised of thiol variants wherein the thiol variants being selected from the group comprising free cysteine variant, cysteinylated variant, cystinylated variant, glutathionylated variant, variants having intramolecular or intermolecular disulfide bond scrambling. In another embodiment, the said anti-IL-17A antibody has a non-canonical cysteine residue in the CDR region of the light chain. In some embodiment, the said anti-IL-17A antibody composition comprises of thiol variants wherein the free sulfhydryl present in the non-canonical cysteines bear same chemical modification or are differently modified e.g. cysteinylation in both the light chains or cysteinylation in one light chain while glutathionylation in other light chain. In yet another embodiment, the thiol variants in the said antibody composition comprises of antibody dimers which result due to formation of disulfide linkage between the free cysteines of two different antibody molecules.

In a preferred embodiment, the said anti-IL-17A antibody has a non-canonical cysteine at 97^{th} position of light chain, the position being designated according to Kabat numbering scheme. In most preferred embodiment, the said antibody is secukinumab.

In one of the embodiment, the said the said anti-IL-17A antibody composition comprises of thiol variants wherein the thiol variants being selected from the group comprising free cysteine variant, cysteinylated variant, cystinylated variant, glutathionylated variant, variants having intramolecular or intermolecular disulfide bond scrambling, antibody dimer variants, wherein the free cysteine variant is the most abundant species present in the composition. In yet another embodiment, the said anti-IL-17A antibody composition comprises of thiol variants wherein the free cysteine variant comprises about 50%-99% free cysteine variant.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### Examples

Commercially available secukinumab samples (Cosentyx/Scapho) and secukinumab samples obtained from CHO cells expressing secukinumab were used for the method described below.

Thiol Variant Analysis at Intact Protein Level

Intact protein mass analysis of anti-IL-17A IgG1 was performed with and without the glycovariants on the protein backbone.

Intactprotein (IP): The anti-IL-17A IgG1 was diluted with milliQ water to a final concentration of 1 mg/mL to analyze the protein backbone with the glycosylation variants.

Intact protein with PNGaseF treatment (IPPF): the anti-IL-17A IgG1 was diluted with milliQ water to a final concentration of 1 mg/mL and treated with PNGase F to analyze the protein backbone devoid of glycosylation variants.

A UPLC system (Waters) with a Waters Bioresolve RP mAb polyphenyl column (2.7 μm 2.1×150 mm, 450 Å) coupled with Xevo G2-XS QTOF mass spectrometer was used to measure the intact molecular weights. The column temperature was set at 80° C. and 1 μL of sample was injected onto the column using given gradient mentioned in Table 1.

TABLE 1
Mobile phase gradient used for UPLC

| | | % Mobile | | % Mobile |
|---|---|---|---|---|
| | Flow | Phase A | % Mobile | Phase C |
| Time | rate | (100% milliQ | Phase B | (1% Formic |
| (min) | (ml/min) | water) | (100% ACN) | Acid in water) |
| 0.00 | 0.20 | 70.00 | 20.00 | 10.00 |
| 0.50 | 0.20 | 70.00 | 20.00 | 10.00 |
| 0.51 | 0.20 | 65.00 | 25.00 | 10.00 |
| 3.61 | 0.20 | 40.00 | 50.00 | 10.00 |
| 4.00 | 0.50 | 0.00 | 90.00 | 10.00 |
| 4.10 | 0.50 | 85.00 | 5.00 | 10.00 |
| 4.60 | 0.50 | 0.00 | 90.00 | 10.00 |
| 4.70 | 0.50 | 85.00 | 5.00 | 10.00 |
| 5.20 | 0.50 | 0.00 | 90.00 | 10.00 |
| 5.30 | 0.50 | 70.00 | 20.00 | 10.00 |
| 6.00 | 0.50 | 70.00 | 20.00 | 10.00 |

UV absorbance for the protein was measured at a wavelength of 280 nm. The mass spectrometer was run in positive mode with the following settings: a scan range of m/z 500-4000, desolvation temperature was set at 350° C., source temperature was set at 120° C., capillary voltage was set at 3 kV, sampling cone voltage was set at 150V and desolvation gas flow was set at 800 L/h. The raw data files were processed with Waters UNIFI (version 1.9.4.053).

Thiol Variant Analysis at Peptide Level

Peptide mapping analysis was performed in non-reduced alkylated as well as non-alkylated samples. To prepare non-reduced alkylated samples (NR-A) samples, 100 μg of the antibody was diluted with denaturation buffer consisting of 8.2 M GdnHCl, 1 mM EDTA and 0.1 M Tris and adjusting the pH to 7.5±0.2 to attain a final protein concentration of 1 mg/mL. The protein solution was mixed and kept at RT for few minutes. Further, alkylation of the protein was carried out by adding L of 0.5 M IAM to the above solution (final concentration ˜10 mM to 20 mM) and incubated at RT. For non-reduced non alkylated (NR-NA) samples, IAM was not added. The NR-NA and NR-A samples were then buffer exchanged into trypsin digestion buffer consisting of 1 M Urea, 1 mM EDTA, 20 mM Hydroxyl ammonium chloride and 0.1 M Tris (pH 7.5±0.2), using a PD-10 gel filtration (GE Healthcare) column. The PD-10 eluate (100 μL) was mixed with trypsin (4 μL, Promega) at a final concentration of 50:1 (protein: enzyme) at 37° C. for 17 hours.

The trypsin digested sample was separated by RP-UPLC (ACQUITY, Waters) on a C18 column (BEH C18, 300 Λ, 1.7 μm, 2.1 mm×150 mm; Waters) and analyzed online with Xevo G2 XS QTOF mass spectrometer. The column temperature was set at 60° C. and 20 μL of sample was injected onto the column using the below given differential gradient (Table 2):

TABLE 2
Mobile phase gradient used for UPLC

| | Flow | % Mobile Phase A | % Mobile | % Mobile |
|---|---|---|---|---|
| Time | rate | (100% milliQ | Phase B | Phase C (1% |
| (min) | (ml/min) | water) | (100% ACN) | TFA in water) |
| 0.0 | 0.3 | 89.0 | 1.0 | 10.0 |
| 1.0 | 0.3 | 89.0 | 1.0 | 10.0 |
| 16.0 | 0.2 | 78.0 | 12.0 | 10.0 |
| 32.0 | 0.3 | 70.0 | 20.0 | 10.0 |
| 61.0 | 0.3 | 50.0 | 40.0 | 10.0 |
| 64.0 | 0.3 | 10.0 | 80.0 | 10.0 |
| 68.0 | 0.3 | 10.0 | 80.0 | 10.0 |
| 68.2 | 0.3 | 89.0 | 1.0 | 10.0 |
| 75.0 | 0.3 | 89.0 | 1.0 | 10.0 |

UV absorption was measured at a wavelength of 214 nm and 280 nm. The mass spectrometer was run in positive mode with the following settings: a scan range of m/z 50-2500, desolvation temperature was set at 350° C., source temperature was set at 120° C., capillary voltage was set at 3 kV, sampling cone voltage was set at 40V and desolvation gas flow was set at 800 L/h. The raw data files were processed with Waters UNIFI (Version 1.9.4.053).

Results:

The sample analysed showed presence of following variants: free cysteine variant—neither of the free sulfhydryls modified, cysteinylated (1 Cys) variant—only one free sulfhydryl cysteinylated, cysteinylated (2 Cys) variant—both the free sulfhydryls cysteinylated, glutathionylated (1 GSH) variant—only one free sulfhydryl glutathionylated, glutathionylated (2 GSH) variant—both the free sulfhydryls glutathionylated, heterogenous thiol variant (1 Cys+1 GSH)—one of the free sulfhydryls cysteinylated while the other glutathionylated, dimer variant—two antibody molecules linked through disulfide linkage formed between the non-canonical cysteines. Due to symmetry of peptides on both light chains, only 3 forms [i.e. free cysteine variant, cysteinylated (1 Cys) variant and glutathionylated (1 GSH) variant] were observed at peptide level. Doublet forms such as cysteinylated (2 Cys) variant and glutathionylated (2 GSH) variant could only be identified at intact protein level. Table 3 tabulates the presence of various variants in the samples analysed.

TABLE 3
Various Thiol variants identified in the secukinumab samples
analyzed.

| | | CHO cell |
|---|---|---|
| | Commercial | expressed |
| Thiol Variant | Secukinumab | Secukinumab |
| Free Cysteine variant (Protein Level) | Present | Present |
| Cysteinylated [1 Cys] | Absent | Present |
| variant (Protein Level) | | |
| Cysteinylated [2 Cys] | Absent | Present |
| variant (Protein Level) | | |
| Glutathionylated [1 GSH] | Present | Present |
| variant (Protein Level) | | |
| Glutathionylated [2GSH] | Present | Present |
| variant (Protein Level) | | |
| Combined [1Cys + 1GSH] | Present | Present |
| variant (Protein Level) | | |
| Free Cysteine variant (Peptide Level) | Present | Present |
| Cysteinylated [1 Cys] | Present | Present |
| variant (Peptide Level) | | |
| Glutathionylated [1 GSH] | Present | Present |
| variant (Peptide Level) | | |
| Dimer variant (Peptide level) | Absent | Present |

1. A method to identify and characterize thiol variants of an antibody in an antibody composition, the method comprising
sample preparation, subjecting the sample to ultra-performance liquid chromatography (UPLC), detecting the variant using high resolution mass spectrometry and identifying the variants
wherein the thiol variants arise due to presence of free cysteines and/or chemical modification of free cysteines derived from either a canonical cysteine involved in disulfide linkage or a non-canonical cysteine.

2. A method to identify and characterize thiol variants of an antibody in an antibody composition, the method comprising
sample preparation, subjecting the sample to ultra-performance liquid chromatography (UPLC), detecting the variant using high resolution mass spectrometry and identifying the variants
wherein the thiol variants arise include variants selected from free cysteine variant, cysteinylated variant, cystinylated variant, glutathionylated variant and dimer variant.

3. The method as in claim 1 wherein the antibody has one or more cysteine residues.

4. An anti-IL-17A antibody composition comprising of thiol variants arising due to presence of free cysteine and/or chemical modification of free cysteine present in said antibody.

5. The antibody composition of claim 4, wherein the said thiol variants comprises of one or more variants selected from group comprising free cysteine variant, cysteinylated variant, cystinylated variant, glutathionylated variant and dimer variants.

6. The antibody composition of claim 4, wherein the said antibody has one or more cysteine residue derived from either a canonical cysteine involved in disulfide linkage or a non-canonical cysteine.

7. The antibody composition according to claim 6, wherein the said non-canonical cysteine residue is in the CDR region of the light chain.

8. The antibody composition according to claim 6, wherein the non-canonical cysteine is present at the 97^{th} position of the light chain, the position being designated according to Kabat numbering scheme.

9. The antibody composition according to claim 4, wherein the said anti-IL-17A antibody is secukinumab.

10. The method as in claim 2 wherein the antibody has one or more cysteine residues.
